(19) United States
(12) Patent Application Publication
(10) Pub. No.: US 2010/0261905 A1
(43) Pub. Date: Oct. 14, 2010

(54) Ruthenium Catalysts for the Production of Hydrocodone, Hydromorphone or a Derivative Thereof

(75) Inventor: David J. Gindelberger, Richmond Heights, MO (US)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 12/757,070

(22) Filed: Apr. 9, 2010

Related U.S. Application Data
(60) Provisional application No. 61/226,302, filed on Jul. 17, 2009.
(60) Provisional application No. 61/167,876, filed on Apr. 9, 2009.

(51) Int. Cl. C07D 491/048

(52) U.S. Cl. 546/45

(57) ABSTRACT

The present disclosure generally relates to catalytic methods for producing opioid derivatives. More particularly, the present disclosure relates to the preparation of hydrocodone, hydromorphone, or a derivative thereof, by means of an isomerization of codeine, morphine, or a derivative thereof, respectively, using a ruthenium catalyst.

### CROSS REFERENCE TO RELATED APPLICATIONS

This application claims the benefit of U.S. Provisional Application No. 61/226,302 filed Jul. 17, 2009, and U.S. Provisional Application No. 61/167,876 filed Apr. 9, 2009, both of which are incorporated herein in their entirety.

### BACKGROUND OF THE DISCLOSURE

The present disclosure generally relates to catalytic methods for producing opioid derivatives. More particularly, the present disclosure relates to the preparation of hydrocodone, hydromorphone, or a derivative thereof, by means of an isomerization of codeine, morphine, or a derivative thereof, respectively, using a ruthenium catalyst.

Hydrocodone and hydromorphone are opiate analgesics having similar qualities to codeine and morphine. Development of new opiate derivatives is desirable to produce new intermediates and potential sources of new analgesics. Conventional methods for producing hydrocodone and hydromorphone typically involve a two step oxidation/reduction route from codeine and morphine, respectively. Unfortunately, these methods can be expensive and inefficient. Attempts to improve efficiency have included the use of catalytic methods. Known catalytic methods include the use of metallic catalysts or complexes, optionally deposited on a support of some kind (e.g., an activated carbon support). However, the preparation of these known catalysts can be difficult. Furthermore, yields are often poor, and isolation of the product is often burdensome. Finally, some catalysts require manufacture and incorporation of expensive supports.

Other known catalytic methods, including the use of finely-divided platinum or palladium in an acidic media, can be environmentally undesirable. Enzymatic methods of conversion have also been attempted. However, like many of the catalysts discussed above, these methods can be costly and difficult to scale up.

Accordingly, a need continues to exist for improved methods for producing various opioids, including hydrocodone, hydromorphone, and derivatives thereof. Desirably, such methods would provide improved yields of the desired reaction product, while enabling the more cost-effective scale up and manufacture of such compounds.

### SUMMARY OF THE DISCLOSURE

Accordingly, it is to be noted that, in one embodiment of the present disclosure, a method for converting a compound of general Formula I to a compound of general Formula II is provided:

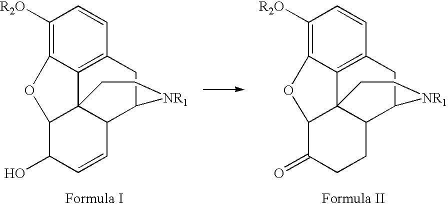

wherein: R₁ is selected from the group consisting of hydrogen and substituted or unsubstituted alkyl, allyl, cycloalkyl, aryl, aryl alkyl, aryl sulfonyl, alkyl sulfonyl, acyl, formyl, hydroxyl, carboxyester and carboxyamide; and, R₂ is selected from the group consisting of hydrogen and substituted or unsubstituted alkyl, aryl, aryl alkyl, acyl, aryl sulfonyl, alkyl sulfonyl, carboxyester, carboxyamide, trialkylsilyl, and heterocycloalkyl. The method comprises contacting the compound of Formula I with a catalyst having a formula RuL_{y}(R₃R₄SO)_{n-y}Xₘ to convert the compound of Formula I to the compound of Formula II. In the catalyst formula: L, when present, is a ligand other than a sulfoxide ligand; R₃ and R₄ are independently selected from the group consisting of alkyl, aryl, alkoxy, or aryloxy; X is a species covalently or non-covalently bound or associated with the remaining portion of the catalyst; m has a value of 1 or 2; y has a value of 0, 1, 2 or 3; and, n has a value of 1, 2, 3 or 4.

In another embodiment, the present disclosure is directed to a method for converting a compound of the Formula III to a compound of Formula IV:

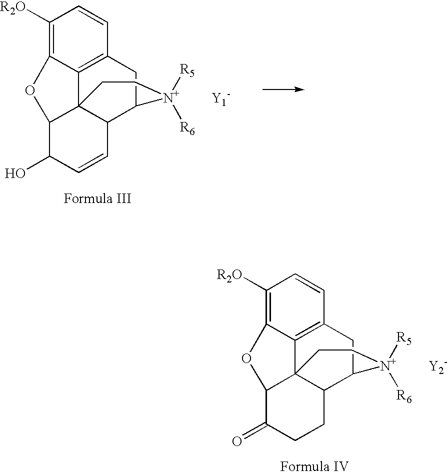

wherein: R₅ and R₆ are independently selected from the group consisting of hydrogen and substituted or unsubstituted alkyl, allyl, cycloalkyl, aryl, aryl alkyl, aryl sulfonyl, alkyl sulfonyl, acyl, formyl, hydroxyl, carboxyester and carboxyamide; R₂ is selected from the group consisting of hydrogen and substituted or unsubstituted alkyl, aryl, aryl alkyl, acyl, aryl sulfonyl, alkyl sulfonyl, carboxyester, carboxyamide, trialkylsilyl, and heterocycloalkyl; and, Y₁ and Y₂ are an anions, which may be the same or different. The method comprises contacting the compound of Formula III with a catalyst having a formula RuL_{y}(R₃R₄SO)_{n-y}Xₘ to convert the compound of Formula III to the compound of Formula IV, wherein: L, when present, is a ligand other than a sulfoxide ligand; R₃ and R₄ are independently selected from the group consisting of alkyl, aryl, alkoxy, or aryloxy; X is a species covalently or non-covalently bound or associated with the remaining portion of the catalyst; m has a value of 1 or 2; y has a value of 0, 1, 2 or 3; and, n has a value of 1, 2, 3 or 4.

In yet another embodiment, the present disclosure is directed to a method for producing a compound of general Formula II:

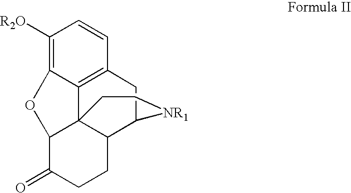

wherein: R₁ is selected from the group consisting of hydrogen and substituted or unsubstituted alkyl, allyl, cycloalkyl, aryl, aryl alkyl, aryl sulfonyl, alkyl sulfonyl, acyl, formyl, hydroxyl, carboxyester and carboxyamide; and, R₂ is selected from the group consisting of hydrogen and substituted or unsubstituted alkyl, aryl, aryl alkyl, acyl, aryl sulfonyl, alkyl sulfonyl, carboxyester, carboxyamide, trialkylsilyl, and heterocycloalkyl. The method comprises: (i) contacting a pre-catalyst of the formula RuL_{y}(R₃R₄SO)_{n-y}Xₘ with an activator to form an activated catalyst of formula RuL_{y}(R₃R₄SO)_{n-y}HXₘ₋₁, wherein: L, when present, is a ligand other than a sulfoxide ligand; R₃ and R₄ are independently selected from the group consisting of alkyl, aryl, alkoxy, or aryloxy; X is a species covalently or non-covalently bound or associated with the remaining portion of the pre-catalyst or activated catalyst; H is a hydrogen atom or ion covalently or non-covalently bound or associated with the remaining portion of the activated catalyst; m has a value of 1 or 2; y has a value of 0, 1, 2 or 3; and, n has a value of 1, 2, 3 or 4; and, (ii) contacting a compound of Formula I with the activated catalyst,

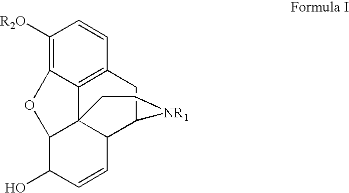

wherein R₁ and R₂ are defined as above, to obtain the compound of Formula

In yet another embodiment, the present disclosure is still further directed to a method for producing a compound of general Formula IV,

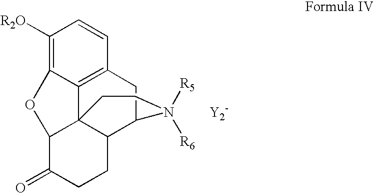

wherein: R₂ is selected from the group consisting of hydrogen and substituted or unsubstituted alkyl, aryl, aryl alkyl, acyl, aryl sulfonyl, alkyl sulfonyl, carboxyester, carboxyamide, trialkylsilyl, and heterocycloalkyl; R₅ and R₆ are independently selected from the group consisting of hydrogen and substituted or unsubstituted alkyl, allyl, cycloalkyl, aryl, aryl alkyl, aryl sulfonyl, alkyl sulfonyl, acyl, formyl, hydroxyl, carboxyester and carboxyamide; and, Y₂ is an anion. The method comprises: (i) contacting a pre-catalyst of the formula RuL_{y}(R₃R₄SO)_{n-y}Xₘ, with a base to form an activated catalyst of formula RuL_{y}(R₃R₄SO)_{n-y}HXₘ₋₁, wherein: L, when present, is a ligand other than a sulfoxide ligand; R₃ and R₄ are independently selected from the group consisting of alkyl, aryl, alkoxy, or aryloxy; X is a species covalently or non-covalently bound or associated with the remaining portion of the pre-catalyst or activated catalyst; H is a hydrogen atom or ion covalently or non-covalently bound or associated with the remaining portion of the activated catalyst; m has a value of 1 or 2; y has a value of 0, 1, 2 or 3; and, n has a value of 1, 2, 3 or 4; and, (ii) contacting a compound of Formula III with the activated catalyst,

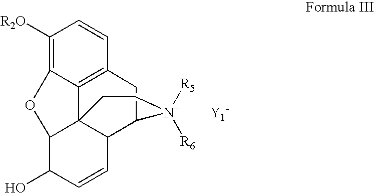

wherein R₂, R₅ and R₆ are defined as above, and Y₁ is an anion, which may be the same as or different than Y₂, to form the compound of Formula IV.

It is to be noted that one or more of the additional features detailed below may be incorporated into one or more of the above-noted embodiments, without departing from the scope of the present disclosure.

### DETAILED DESCRIPTION OF THE DISCLOSURE

In accordance with the present disclosure, it has been discovered that a ruthenium-based or ruthenium-containing catalyst, and more particularly a ruthenium-sulfoxide-based or ruthenium-sulfoxide-containing catalyst, may be used in a method for producing opioid derivatives. In one or more preferred embodiments, the catalyst may be used in the preparation of hydrocodone, hydromorphone, or a derivative thereof, by means of a catalyzed isomerization of codeine, morphine, or a derivative thereof, respectively.

The catalysts detailed herein have been found to possess high activity toward such isomerization reactions. As an illustration, and therefore not to be viewed in a limiting sense, in various embodiments a conversion of at least about 85 mole %, 90 mole %, 95 mole %, 98 mole % or more may be achieved in accordance with the method of the present disclosure, ultimately leading to a compound having a purity of about 90 mole %, 95 mole %, 98 mole % or more (as determined by means generally known in the art). In addition, in various embodiments the catalysts may be used in heterogeneous or homogeneous (e.g., supported) forms, as further detailed elsewhere herein.

In this regard it is to be noted that, as used herein, a “ruthenium-based” or “ruthenium-containing” catalyst, as well as a “ruthenium-sulfoxide-based” or “ruthenium-sulfoxide-containing” catalyst, refers to a catalyst that includes ruthenium as the metal that is complexed (e.g., bound or associated) by or with various ligands (or other species or moieties), including a sulfoxide ligand.

1. Opioid Starting Materials and Isomerization Products

In one embodiment of the present disclosure, a compound of general Formula I may be contacted with a ruthenium-based catalyst in an isomerization reaction to produce a compound of general Formula II:

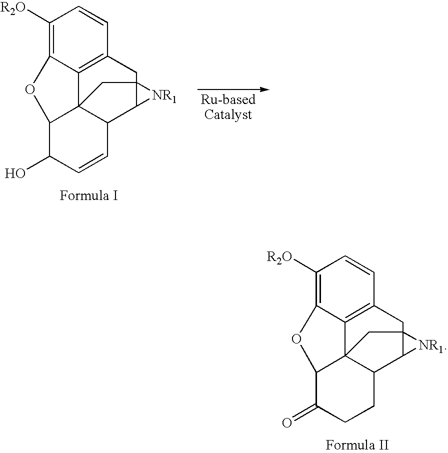

In the structures, R₁ may be selected from, for example, hydrogen and substituted or unsubstituted alkyl, allyl, cycloalkyl, aryl, aryl alkyl, aryl sulfonyl, alkyl sulfonyl, acyl, formyl, hydroxyl, carboxyester and carboxyamide. Additionally, R₂ may be selected from, for example, hydrogen and substituted or unsubstituted alkyl, aryl, aryl alkyl, acyl, aryl sulfonyl, alkyl sulfonyl, carboxyester, carboxyamide, trialkylsilyl, and heterocycloalkyl (e.g., tetrahydropyranyl or tetrahydrofuranyl). In several particular embodiments, R₁ is methyl and R₂ is methyl or H; that is, Formula I may be codeine or morphine, respectively, leading to the formation of hydrocodone or hydromorphone, respectively. Stated differently, the conversion reaction may be carried out to convert a compound of Formula IA to a compound of Formula IIA, or it may be carried out to convert a compound of Formula IB to a compound of Formula IIB, as illustrated below:

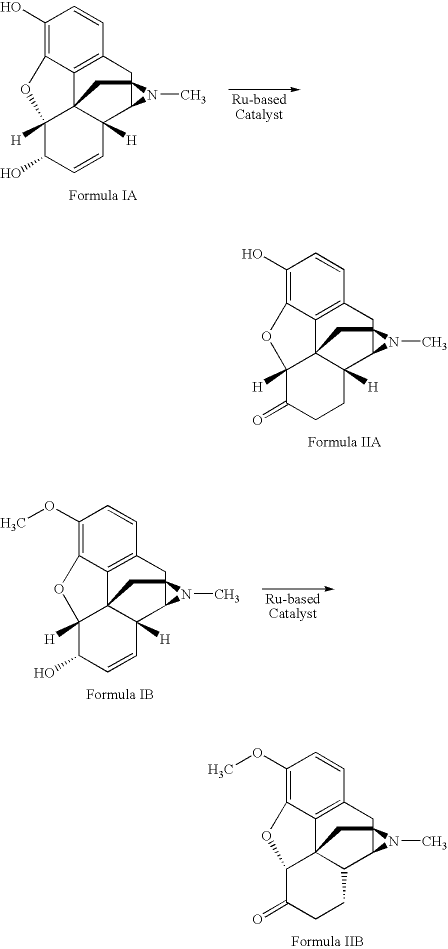

In an alternative embodiment, however, the reactant or starting compounds and/or reaction products may be in the form quaternary amine (or ammonium) salts. For example, in such an embodiment, a compound of general Formula III may be contacted with a ruthenium-based catalyst in an isomerization reaction to produce a compound of general Formula IV:

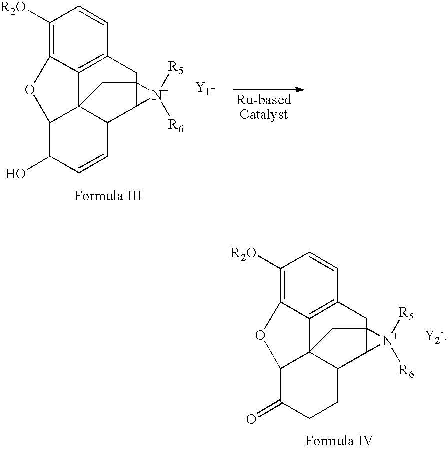

In the structures, R₂ is defined as above, while and R₅ and R₅ may be, for example, independently selected from hydrogen, and substituted or unsubstituted alkyl, aryl, aryl alkyl, acyl, aryl sulfonyl, alkyl sulfonyl, carboxyester and carboxyamide. Y₁ and Y₂ are anions, each independent selected from, for example, a halogen ion (e.g., Cl₋, F₋, Br₋, I⁻), as well as H⁻, BF₄⁻, PF₆⁻, CIO₄⁻, CHO₂⁻, CF₃CO₂⁻, CF₃SO₃⁻, CH₃CO₂⁻, ArCO₂, CH₃SO₃⁻, p-tolylSO₃, HSO₄⁻ and H₂PO₄⁻. It is to be noted that Y₁ and Y₂ may be the same or different, and may be exchanged with the counter ions of the catalyst during the isomerization reaction; that is, in various embodiments Y₁ may be exchanged with a catalyst counter ion Xₘ⁻ (as described elsewhere herein), which is represented as Y₂ in Formula IV.

It is to be further noted that while the starting compounds, and isomerization reaction product compounds, illustrated above have the same base or core structure (i.e., a fused, tetracyclic structure), the methods of the present disclosure may be used with essentially any alkaloid having an allyl alcohol functionality. Additionally, or alternatively, it is to be noted that while the base or core structure of the compounds illustrated above have a specific arrangement of substituents, additional substituents and/or different substituents may be present at one or more sites therein without departing from the scope of the present disclosure, provided the substituted structure remains an alkaloid having an allyl alcohol functionality therein. For example, in one or more alternative embodiments the starting compound and resulting isomerization reaction product compound may have the structures of Formulas V and VI, respectively:

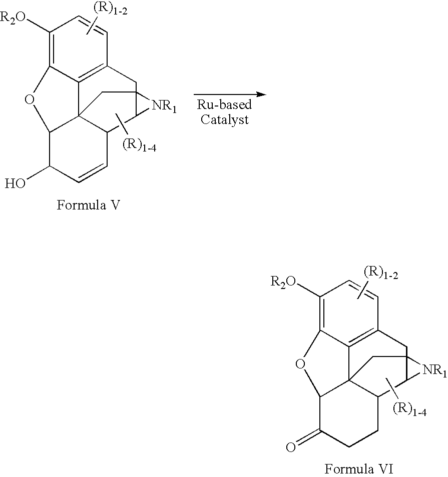

wherein each R substituent may be the same or different in the starting compound structure and the isomerization reaction product structure, and may be independently selected from, for example, substituted or unsubstituted alkyl, allyl, cycloalkyl, aryl, aryl alkyl, aryl sulfonyl, alkyl sulfonyl, acyl, formyl, hydroxyl, carboxyester and carboxyamide (the other variables in the structures being as previously defined above). Accordingly, the structures illustrated in, for example, Formulas I through IV above should not be viewed in a limit sense.

In is to be still further noted that the starting compounds referenced herein, such as those of Formula I, Formula III and Formula V, and in particular the compounds of Formula IA and Formula IB, may be obtained commercially, and/or may be prepared according to methods generally known in the art, including for example the methods disclosed in U.S. Pat. No. 7,495,098, the entire contents of which are incorporated herein by reference for all relevant and consistent purposes.

2. Ruthenium-Based Catalysts

In accordance with the present disclosure, a compound (such as a compound of the general Formula I, Formula III or Formula V) is contacted with a ruthenium-based catalyst having a formula: RuL_{y}(R₃R₄SO)_{n-y}Xₘ. L, when present, is a ligand other than a sulfoxide ligand; that is, L is a non-sulfoxide ligand (and thus has a structure other than, for example, R₃R₄SO). R₃ and R₄ are independently selected from substituted or unsubstituted alkyl, aryl, alkoxy, and aryloxy. X is a species covalently or non-covalently bound or associated with the remaining portion of the catalyst. Finally, m, y and n are integers, wherein: m has a value of 1 or 2; y has a value of 0, 1, 2 or 3; and, n has a value of 1, 2, 3 or 4. The net or overall charge of the ruthenium-based catalyst or complex is typically zero. However, in various embodiments the net or overall charge of the catalyst or complex may be other than zero without departing from the scope of the present disclosure, the catalyst or complex having, for example, a net +1 charge (such as, for example, when the catalyst or complex has the formula [Ru(DMSO)₆]⁺²[BPh₄]₂⁻¹ or the formula [RuL₂(DMSO)₄]⁺²[BPh₄]₂).

In one particular embodiment, y may be 0 and n may be 4, which means the ligand L is not present. Thus, the catalyst may have the general formula Ru(R₃R₄SO)₄Xₘ, wherein R₃, R₄, X and m are as defined elsewhere herein. In various alternative embodiments, however, y may be 1, 2 or 3. In those embodiments, each L present may be the same or different and may be independently selected from, for example, H, H₂, CO, and the general formula (PR₇R₈R₉), wherein R₇, R₈, and R₉ may be independently selected from, for example, substituted or unsubstituted alkyl, aryl, alkoxy, and aryloxy. In one particular embodiment, L is (PPh₃), while in other particular embodiments L is CO, H, or H₂.

In these or other particular embodiments, R₃ and/or R₄ may be the same or different and may be independently selected from alkyl (e.g., lower alkyl, having from 1 to about 10 carbon atoms, or from about 1 to about 6 carbon atoms, selected from for example methyl, ethyl, propyl, butyl, pentyl or hexyl), aryl (e.g., phenyl), alkoxy (e.g., lower alkoxy, having from 1 to about 10 carbon atoms, or from about 1 to about 6 carbon atoms) or aryloxy (e.g., phenoxy). In one preferred embodiment, however, R₃ and/or R₄ are methyl groups.

As stated above, X is a species (i.e., an atom, or alternatively a moiety or substituent) that is covalently bound to, or non-covalently bound to or associated with (through, for example, ionic interactions), the remaining portion of the catalyst, and the ruthenium metal atom in particular. In various embodiments, X may be in the form of a suitable counter ion (e.g., an anion). In one or more preferred embodiments, X may be a halogen (e.g., Cl, F, Br, or I, or an anion thereof), or an anion selected from the group consisting of H⁻, BF₄⁻, PF₆⁻, CIO₄⁻, CHO₂⁻, CF₃CO₂⁻, CF₃SO₃⁻, CH₃CO₂⁻, ArCO₂, CH₃SO₃⁻, p-tolylSO₃, HSO₄⁻ and H₂PO₄⁻, and tertiary borates, such as B(Ar)₄⁻¹. In one or more preferred embodiments, X may be Br or Cl (or an anion thereof).

Also as stated above, m may be 1 or 2. When m is 2, it is to be noted that X may in some embodiments be the same (i.e., X₂), such as for example Br₂, Cl₂, F₂, 1₂, or H₂. Alternatively, when m is 2, X₂ may have the general formula X′X″, wherein X′ and X″ are a different species (e.g., different counter ions), such as for example X′=H and X″=Cl or Br. It is therefore to be noted that, in such embodiments, each X may be independently selected from the list provided above, such that the present disclosure extends to essentially any combination or permutation possible therein.

In this regard it is to be further noted that X may be essentially any species (e.g., counter ion) known in the art to be suitable for such a use. Accordingly, the species provided herein should not be viewed in a limiting sense.

It is to be still further noted that, with regard to the compounds of Formulas III and IV, in some embodiments X may be exchanged with Y₁ during the isomerization reaction; that is, in some reactions X and Y₂ may be the same.

In view of the foregoing, it is to be noted that, in various preferred embodiments, the catalyst may have a formula selected from, for example: Ru(DMSO)₄Cl₂, Ru(PPh₃) (DMSO)₃Cl₂, Ru(PPh₃) (DMSO)₃Cl₂, Ru(DMSO)₄Br₂, Ru(PPh₃) (DMSO)₃Br₂, and Ru(PPh₃) (DMSO)₃Cl₂. In various alternative embodiments, wherein at least one X is hydrogen, the catalyst may have a formula selected from, for example: Ru(DMSO)₄H₂, Ru(DMSO)₄HCl, Ru(DMSO)₄HBr, Ru(PPh₃) (DMSO)₃H₂, Ru(PPh₃) (DMSO)₃HCl and Ru(PPh₃) (DMSO)₃HBr.

One or more of the catalysts described herein may be activated prior to contact with the reaction starting materials (e.g., obtained in activated form ready for use), or may be activated as part of the reaction process (i.e., obtained in an inactive form and activated prior to or concurrently with the isomerization reaction), using methods generally known in the art, including for example methods by which a hydride-containing form of the catalyst is prepared (as disclosed, for example, by B. N. Chaudret et al., “The Reactions of Chlorohydrido- and Dichloro-tris(triphenylphosphine)-rutheniurn(II) with Alkali Hydroxides and Alkoxides,” J. Chem. Soc. Dalton, pp. 1546-1557 (1977)). For example, in one or more embodiments herein the catalyst may be contacted with a suitable activator (e.g., a base, such as a Lewis base) in order to render it active for use in the isomerization reaction. Accordingly, in those instances wherein the catalyst requires activation, it may be optionally referred to herein as a “pre-catalyst” before activation and an “activated catalyst” once activation has been achieved.

In one or more alternative embodiments, the catalyst (i.e., pre-catalyst or activated catalyst), may be obtained or used in an oligomeric (i.e., cluster) or polymeric form. For example, the catalyst may be obtained or used in a form characterized by the formula [RuL_{y}(R₃R₄SO)_{n-y}Xₘ]ₚ, wherein p has a value of more than 1 (e.g., 2, 4, 6, 8, 10 or more). Exemplary oligomeric (or cluster) forms of the catalyst include Ru₂Cl₄(DMSO)₅, as well as catalysts having the generally formula [cation]ₘ₋₂[RuL_{y}(R₃R₄SO)_{n-y}Xₘ], wherein “[cation]” generally references essentially any known cationic species suitable for use in accordance with the present disclosure, including for example ammonium and sodium cations.

The catalysts, or pre-catalysts, may be prepared by methods knows in the art, and/or many be obtained commercially, including those in polymeric or oligomeric form. For example, methods for the generation of one or more ruthenium-based complexes suitable for use as a catalyst (or pre-catalyst) in accordance with the present disclosure are generally described by I. P. Evans et al. (“Dichlorotetrakis(dimethyl sulphoxide) ruthenium(II) and its Use as a Source Material for Some New Ruthenium(II) Complexes,” J. Chem. Soc., Dalton Trans., 204-209 (1973)), and/or T. Bora et al. (“Some Dimethyl Sulphoxide and Sulphide Complexes of Ruthenium,” J. lnorg. Nucl. Chem., vol. 38, 1815-1820 (1976)), as well as G. A. Heath et al. (“The Structural Reformulation of [Ru₂Cl₄(Me₂S0)₅],” J. Chem. Soc. Dalton Trans., 2429-2432 (1982), wherein ruthenium-based complex clusters are disclosed).

3. Catalyst Supports

As previously noted, the ruthenium-based catalysts of the present disclosure may be homogeneous, or they may be heterogeneous and include a solid support. Suitable catalyst supports include, for example, alumina, silica (including functionalized silica supports), yttria, zeolite, siloxanes (e.g., —Si(OR)—O—)ₙ, which may be useful for example in biphasic systems to help keep the catalyst separate from the reaction product), or a suitable polymer. In one particular embodiment, wherein the catalyst includes a phosphine, such as a tertiary phosphine, the phosphine itself may be solid supported. In this or an alternative embodiment, one of the R groups of the catalyst (e.g., R₅ or R₆ or R₇) may contain a linking group connecting the phosphine to the solid phase, as is well known in the art.

Many solid supported tertiary phosphines are commercially available, or may be prepared by methods generally known in the art. For example, one non-limiting example of a solid supported tertiary phosphine that may be used in accordance with the present disclosure is a silica supported tertiary phosphine made from treating silica with (EtO)₃SiCH₂CH₂PPh₂. Another non-limiting example of a solid supported tertiary phosphine is the copolymer prepared from the polymerization of the monomer p-styryldiphenylphosphine, also known as diphenyl(p-vinylphenyl)phosphine and having the formula:

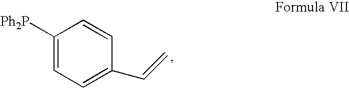

with styrene. Optionally, other monomers may be substituted or added to optimize certain physical properties of the polymeric catalyst. Illustrative examples include, but are not limited to, ethylene dimethacrylate, p-bromostyrene, and crosslinking agents such as divinylbenzene, butadiene, diallyl maleate, diallyl phthalate, glycol dimethacrylate, and other di- or triolefins. Other phosphine containing monomers bound to the styrene ring may have, in addition to diaryl substitutions, dialkyl, branched and cyclic dialkyl, dialkoxyl or mixed substitutions of these compounds.

The polymeric support may, for example, be composed of a styrene divinylbenzene copolymer containing about 2 mole % to about 20 mole % divinylbenzene and about 75 mole % to about 97.5 mole % styrene. Additionally, about 0.5 mole % or about 1 mole % to about 7 mole %, or about 5 mole % to about 6 mole %, of the pendant phenyl groups from the copolymerized styrene may contain the diphenylphosphine moiety at the para position (e.g., p-diphenylphosphenostyrene). The Ru/pendant atom ratio may be at least about 0.001, about 0.01, about 0.1, or even at least about 0.5. The upper limit may be set by the point at which the polymer support will no longer take up the complex, the upper limit being for example about 1.2.

The polymeric complex may be made by contacting a solution of the ruthenium-base catalyst, or ruthenium salt complex, in solution with the polymer support, or alternative with any suitable support material. The ruthenium-base catalyst, or ruthenium salt complex, may be dissolved in essentially any suitable solvent, including but not limited to water, methanol, ethanol, isopropanol, isobutyl alcohol, t-butyl alcohol, chloroform, dichloromethane, fluorobenzene, chlorobenzene, toluene, N-methylpyrrolidone, N,N-dimethylformamide, N,N-dimethylacetamide, methyl sulfoxide, methyl sulfone, tetrahydrofuran and mixtures thereof. The complex solution may be at least 10⁻⁶ M in the ruthenium complex, and in some embodiments at least about 10⁻³ M in the ruthenium complex. The resulting load of the ruthenium-base catalyst, or ruthenium salt complex, on the support may vary (as a function of, for example, support type, size, porosity or surface area, solvent, and/or catalyst composition), but may be for example about 2 wt %, about 4 wt %, about 6 wt %, about 8 wt % or more.

Polymer-supported catalysts may be intrinsically porous, thereby imparting increased activity to the catalyst. With catalysts having an organic polymer portion which is not intrinsically porous, porosity may be induced into the polymer portion by solvent swelling. Combinations of the solvents discussed below may be manipulated to produce various degrees of swelling of the polymer portion of the catalyst, as is well known in the art.

4. Isomerization Reaction

The isomerization reaction may be performed according to methods generally known in the art, which involve contacting a starting compound as detailed herein (i.e., a compound of Formula I, Formula III or Formula V) with a catalyst of the present disclosure. An exemplary method includes contacting (e.g., dissolving or suspending) the starting compound in a suitable solvent in a reaction vessel. Suitable solvents may be selected from, for example, known aromatic solvents (e.g., benzene, toluene), hydrocarbon solvents (e.g., pentane, hexane), ethers (e.g., diethyl ether), alcohols (e.g., methanol, ethanol) or diols, as well as known heterocycles (e.g., N-methylpyrrolidone) and amides (e.g., hexamethylphosphoramide). In particular, suitable alcohols include, for example, C₁ to C₁₀ alcohols and dials. In one or more preferred embodiments, the solvent is ethanol.

The reaction vessel may be flushed with an inert atmosphere, such as argon or nitrogen, prior to the addition of the catalyst thereto. The resulting reaction mixture may be refluxed, optionally under the inert atmosphere, until the isomerization reaction (or conversion) is essentially complete (as determined using means generally known in the art, such as for example HPLC, to analyze or measure the concentration of the desired reaction product, and/or the starting compound, in the reaction mixture).

Typically, the molar amount of catalyst added to the reaction (or reaction mixture) may be less than about 10 mole % and more than about 0.1 mole %, as compared to the molar amount of starting compound (e.g., the compounds Formula I, Formula III or Formula V) present in the reaction mixture. For instance, the molar amount of catalyst added to the reaction mixture may be from about 0.1 mole % to about 10 mole % of the molar amount of compounds of Formulas I, III or V present therein, or from about 0.5 mole % or about 1 mole % to about 5 mole %, or from about 1 mole % to about 2 mole %. In this regard it is to be noted, however, that the mole % of the catalyst may be altered as needed in order to optimize yield or conversion, and/or purity, of the desired reaction product. Accordingly, the ranges provided herein are for illustration, and therefore should not be viewed in a limiting sense.

The duration, reaction temperature, reaction pressure, and/or concentration of starting components or reagents in the reaction mixture, may also be altered in order to optimize yield or conversion, and/or purity, of the desired reaction product. Typically, however, the reaction is allowed to continue for at least about 30 minutes and may continue for about 24 hours or more, although in various alternative embodiments the reaction may be allowed to continue for about 1 to about 16 hours, about 2 to about 12 hours, or about 4 hours to about 8 hours. In these or yet other alternative embodiments, the reaction mixture may be maintained at a temperature of from about 0° C. to about 120° C., or about 20° C. to about 110° C., or about 40° C. to about 100° C., or about 60° C. to about 80° C., with one preferred embodiment being carried out under reflux conditions (e.g., about 75° C.), the reaction typically being carried out under atmospheric pressure. In these or yet other alternative embodiments, the concentration of the starting alkaloid compound in the reaction mixture is typically about 1 g per about 5 to about 10 ml of solvent.

In various embodiments, a conversion of at least about 85 mole %, about 90 mole %, about 95 mole %, about 98 mole % or more may be achieved in accordance with the method of the present disclosure, ultimately leading to a compound having a purity of about 90 mole %, about 95 mole %, about 98 mole % or more (as determined by means generally known in the art), after isolation and purification of the reaction product (using means generally known in the art).

Catalyst molar loading rates of about 1% of the molar amount of compounds of Formula I, III or V (i.e., about 1 mole % relative to the molar amount of the starting compound) may result in a conversion of at least about 90 mole % after about 8 hours, while loading rates of about 5% of the molar amount of reactant may result in a conversion of at least about 90 mole % after about 1 hour. Product purity in such embodiments, upon isolation and purification (using means generally known in the art), may be at least about 90 mole %, while purities of at least about 95 mole % or even at least about 98 mole % may be achieved.

A tertiary amine such as, for example, triethylamine, may optionally be added to the reaction mixture. In various embodiments, the addition of the tertiary amine may help reduce the formation of unwanted side products, such as for example the alkaloid neopine, which is a potential side product in reactions of the present disclosure. Additionally, or alternatively, a tertiary amine may be added to serve as a co-catalyst, aiding in the forming of for example a ruthenium hydride species.

After the reaction as reached a desired point of completion (determined as noted above), the mixture may be cooled as needed and the reaction product isolated using methods generally known in the art (e.g., filtration, centrifugation, crystallization, etc.). Once isolated, the reaction product may be further purified if needed, again using methods generally known in the art (e.g., purified by recrystallization in a suitable solvent as is well known in the art, or by any other conventional methods of purification). In some embodiments, the concentration of ruthenium in the product may be controlled to be less than about 12 ppm, about 10 ppm, about 8 ppm, about 6 ppm, or even about 4 ppm, by weight.

The isomerization or conversion reactions of the present disclosure may be carried out in continuous or batch form. The supported catalysts of the present disclosure may, for example, be placed in a column or container as part of a loop reactor. A solution containing compounds of Formula I, III or V may be pumped or gravity fed through a catalyst bed and cycled back to the reactor until the desired conversion to a compound of Formula II, IV or VI, respectively, is produced. This allows many cycles (perhaps several batches) of product to be obtained with a given bed. The catalyst may be easily recovered and reused (directly or after purification or regeneration). In such embodiments, the purification method may be simplified, as the catalyst is not present in the solution. Upon cooling, the product may crystallize out of reaction solution in high purity, to be recovered by filtration or centrifugation.

5. Definitions

The compounds described herein may have asymmetric centers. Compounds of the present disclosure containing an asymmetrically substituted atom may be isolated in optically active or racemic form. All chiral, diastereomeric, racemic forms and all geometric isomeric forms of a structure are intended, unless the specific stereochemistry or isomeric form is specifically indicated. All processes used to prepare compounds of the present disclosure and intermediates made therein are considered to be part of the present disclosure.

As used herein, “optional” or “optionally” means that the subsequently described event or circumstance may or may not occur, and that the description includes instances where the event or circumstance occurs and instances where it does not.

The terms “aryl” or “ar” as used herein, alone or as part of another group, denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halogen” or “halo” as used herein alone or as part of another group refer to chlorine, bromine, fluorine, and iodine.

Unless otherwise indicated, the “alkyl” groups described herein are preferably lower alkyl containing from one to about 10 carbon atoms in the principal chain, and up to about 20 carbon atoms. They may be straight or branched chain or cyclic (e.g., cycloalkyl) and include methyl, ethyl, propyl, isopropyl, butyl, pentyl, hexyl and the like. Accordingly, the phrase “C₁₋₂₀ alkyl” generally refers to alkyl groups having between about 1 and about 20 carbon atoms, and includes such ranges as about 1 to about 15 carbon atoms, about 1 to about 10 carbon atoms, or about 1 to about 5 carbon atoms, while the phrase “C₁₋₁₀ alkyl” generally refers to alkyl groups having between about 1 and about 10 carbon atoms, and includes such ranges as about 1 to about 8 carbon atoms, or about 1 to about 5 carbon atoms.

The term “substituted” as in “substituted aryl” or “substituted alkyl” and the like, means that in the group in question (i.e., the aryl, the alkyl, or other moiety that follows the term), at least one hydrogen atom bound to a nitrogen atom or carbon atom, respectively, is replaced with one or more substituent groups such as hydroxy, alkoxy, amino, halo, and the like. When the term “substituted” introduces a list of possible substituted groups, it is intended that the term apply to every member of that group. That is, the phrase “substituted alkyl, aryl, acyl, etc.” is to be interpreted as “substituted alkyl, substituted aryl, and substituted acryl”, respectively. Similarly, “optionally substituted alkyl, aryl and acyl” is to be interpreted as “optionally substituted alkyl, optionally substituted aryl and optionally substituted acyl.”

The modifiers “hetero”, as in “heterocycle” refer to a molecule or molecular fragment in which one or more carbon atoms is replaced with a heteroatom. Thus, for example, the term “heteroalkyl” refers to an alkyl group that contains a heteroatom, while “heterocycloalkyl” reference to a cycloalkyl group that contains a heteroatom. When the term “heteroatom-containing” introduces a list of possible heteroatom-containing groups, it is intended that the term apply to every member of that group.

As illustrated below, the term “fused, tetracyclic” generally refers to a compound that includes four rings therein, and further wherein each of the rings in the compound share two ring atoms (e.g., carbon atoms or heteroatoms, as highlighted by the dashed-circles below). Optionally, when a heteroatom is present, the “fused hetero-tetracyclic” may be used.

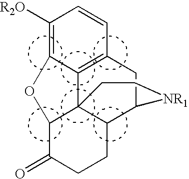

Having described the disclosure in detail, it will be apparent that modifications and variations are possible without departing from the scope of the disclosure defined in the appended claims.

### EXAMPLES

The following non-limiting examples are provided to further illustrate the present disclosure.

### Example 1

### Catalyst Preparation

In this Example the catalyst RuCl₂(DMSO)₄ was prepared as follows: The reactor or reaction vessel (a 100 ml, 3-neck round-bottom flask) was equipped with N₂ inlet, thermocouple, and a condenser capped with a bubbler. A nitrogen atmosphere was the established therein. A charge of 50 ml of ethanol, followed by 5 g (24.2 mmol) RuCl₃.H₂O, was then placed therein. The mixture was refluxed 2 hours. DMSO was then added (7.5 g, 96.6 mmol), resulting in a slight exothermic reaction. The color of the reaction mixture was observed to change from dark green to dark yellow. Reflux of the reaction mixture was continued for an additional hour after DMSO addition.

After reflux, the resulting solution was a bright orange color. The solution was cooled and volatiles removed in vacuo. Acetone (50 ml) was added and the mixture stirred briefly, and then volatiles were removed again (in vacuo). A second 50 ml aliquot of acetone was added, and the solution was warmed and mixed at 60° C. while exposed to a slight vacuum. Large yellow-orange crystals formed. The solution was cooled and the solids collected. The solids were washed with acetone and dried in vacuo. Approximately 9.9 g of product was collected (approximately 85% yield).

### Example 2

### Hydromorphone Preparation (Typical Catalyst Loading)

A sample of hydromorphone was prepared as follows (wherein a typical catalyst loading was used and the crude catalyst salt was isolated essentially immediately after the reaction was complete): A 100 ml 3-neck round-bottom flask equipped with heating mantle, stir bar, N₂ inlet, thermocouple, and condenser capped with a bubbler was used as the reaction or reaction vessel in the reaction. A nitrogen atmosphere was established therein, and then the reactor was charged with 20 ml ethanol. The temperature was set to 80° C. then RuCl₂(DMSO)₄ (0.304 g) and potassium tert-butoxide (0.350 g) were charged thereto. The reaction mixture was heated until reflux began, and then morphine (3.79 g) was added. The reaction progress was followed by TLC (MeOH:NH₄OH 98:2). After approximately 1.5 hours, the reaction appeared to be complete. The reaction mixture was cooled and 1 ml of concentrated HCl (aqueous) was added. The resulting slurry was cooled to 10° C., and then filtered to isolate/collect the crude reaction product (approximately 4.2 g of hydromorphone HCl).

### Example 3

### Hydromorphone Preparation (Low Catalyst Loading)

A sample of hydromorphone was prepared as follows (wherein a catalyst loading below the typical loaded of Example 2 was used and the crude catalyst salt was isolated essentially immediately after the reaction was complete): A 100 ml 3-neck round-bottom flask equipped with heating jacket, overhead mechanical stirrer, N₂ inlet, thermocouple, and condenser capped with a bubbler was used as the reaction or reaction vessel in the reaction. A nitrogen atmosphere was established therein, and then the reactor was charged with 200 ml ethanol. The jacket temperature was set to 90° C. then potassium tert-butoxide (0.60 g) followed by RuCl₂(DMSO)₄ (0.66 g) were charged thereto. The reaction mixture was stirred/mixed for 5 minutes and then morphine (18.09 g, 72% API) was added. The reaction progress was followed by TLC (MeOH:NH₄OH 98:2). After approximately 4 hours, the reaction appeared to be complete, The reaction mixture was allowed to cool, and then the reaction mother liquor was sampled. HPLC analysis showed complete consumption of morphine (the mixture containing approximately 76% hydromorphone HCl). The reaction mixture was filtered to isolate/collected the crude reaction product.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above apparatus and methods without departing from the scope of the disclosure, it is intended that all matter contained in the above description and shown in the accompanying figures shall be interpreted as illustrative and not in a limiting sense.

What is claimed is:

1. A method for converting a compound of the general Formula I to a compound of general Formula II: 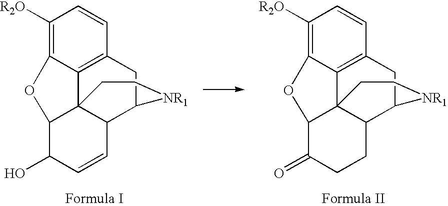
the method comprising contacting the compound of Formula I with a catalyst having a formula RuL_{y}(R₃R₄SO)_{n-y X}ₘ to convert the compound of Formula Ito the compound of Formula II, wherein:
L, when present, is a ligand other than a sulfoxide ligand;
R₃ and R₄ are independently chosen from alkyl, aryl, alkoxy, or aryloxy;
X is a species covalently or non-covalently bound or associated with the remaining portion of the catalyst;
m has a value of 1 or 2;
y has a value of 0, 1, 2 or 3;
n has a value of 1, 2, 3 or 4;
and further wherein:
R₁ is chosen from hydrogen and substituted or unsubstituted alkyl, allyl, cycloalkyl, aryl, aryl alkyl, aryl sulfonyl, alkyl sulfonyl, acyl, formyl, hydroxyl, carboxyester and carboxyamide; and,
R₂ is chosen from hydrogen and substituted or unsubstituted alkyl, aryl, aryl alkyl, acyl, aryl sulfonyl, alkyl sulfonyl, carboxyester, carboxyamide, trialkylsilyl, heterocycloalkyl.

2. The method of claim 1, wherein the conversion reaction is carried out to convert the compound of Formula IA to the compound of Formula IIA:

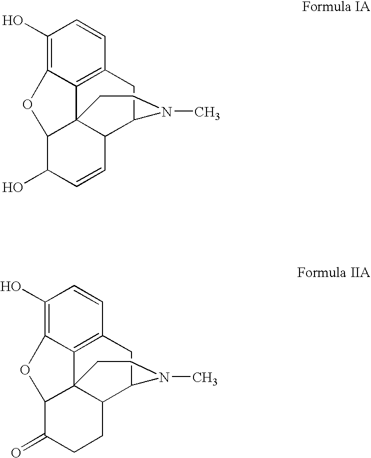

3. The method of claim 1, wherein the conversion reaction is carried out to convert the compound of Formula IB to the compound of Formula IIB:

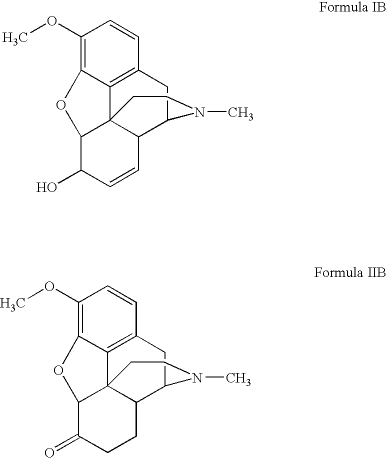

4. The method of claim 1, wherein the catalyst has the formula Ru(R₃R₄S0)₄Xₘ.

5. The method of claim 1, wherein y has a value of 1, 2 or 3, and further wherein each L present is independently chosen from the general formula (PR₅R₆R₇), each of R₅, R₆, and R₇ being independently chosen from alkyl, aryl, alkoxy, and aryloxy.

6. The method of claim 1, wherein R₃ and R₄ are independently chosen from C₁ to C₆ alkyl, aryl, alkoxy, and aryloxy; and each X is independently chosen from a halogen anion and an anion chosen from H⁻, BF₄⁻, PF₆⁻, CIO₄⁻, CHO₂⁻, CF₃CO₂⁻, CF₃SO₃⁻, CH₃CO₂⁻, ArCO₂, CH₃SO₃⁻, p-tolylSO₃, HSO₄⁻ and H₂PO₄⁻ and B(Ar)₄⁻.

7. The method of claim 1, wherein the catalyst is chosen from Ru(DMSO)₄Cl₂, Ru(DMSO)₄H₂, Ru(DMSO)₄HCl, Ru(PPh₃) (DMSO)₃Cl₂, Ru(PPh₃) (DMSO)₃H₂, and Ru(PPh₃) (DMSO)₃HCl.

8. The method of claim 1, wherein the method further comprises activating the catalyst by contacting the catalyst with an activator, and then contacting the activated catalyst in the reaction mixture with the compound of Formula I.

9. The method of claim 8, wherein the catalyst has the formula RuL_{y}(R₃R₄SO)_{n-y}Xₘ prior to activation, and the activated catalyst has the formula RuL_{y}(R₃R₄SO)_{n-y}HXₘ₋₁, wherein H is a hydrogen atom or ion covalently or non-covalently bound or associated with the remaining portion of the activated catalyst.

10. A method for converting a compound of the Formula III to a compound of Formula IV: 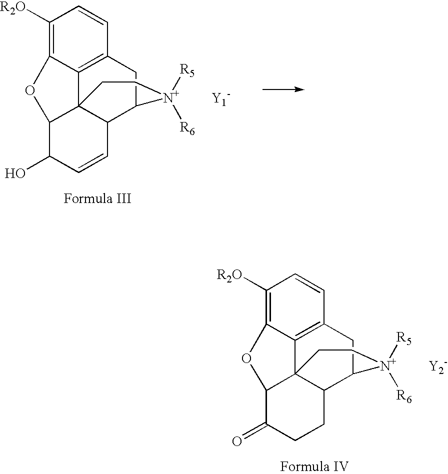
the method comprising contacting the compound of Formula III with a catalyst having a formula RuL_{y}(R₃R₄SO)_{n-y}Xₘ to convert the compound of Formula III to the compound of Formula IV, wherein:
L, when present, is a ligand other than a sulfoxide ligand;
R₃ and R₄ are independently chosen from alkyl, aryl, alkoxy, or aryloxy;
X is a species covalently or non-covalently bound or associated with the remaining portion of the catalyst;
m has a value of 1 or 2;
y has a value of 0, 1, 2 or 3;
n has a value of 1, 2, 3 or 4;
and further wherein:
R₅ and R₆ are independently chosen from hydrogen and substituted or unsubstituted alkyl, allyl, cycloalkyl, aryl, aryl alkyl, aryl sulfonyl, alkyl sulfonyl, acyl, formyl, hydroxyl, carboxyester and carboxyamide;
R₂ is chosen from hydrogen and substituted or unsubstituted alkyl, aryl, aryl alkyl, acyl, aryl sulfonyl, alkyl sulfonyl, carboxyester, carboxyamide, trialkylsilyl, and heterocycloalkyl; and,
Y₁ and Y₂ are each an anion, which may be the same or different.

11. The method of claim 10, wherein Y₁ and Y₂ are independently selected from a halogen anion or an anion chosen from H⁻, BF₄⁻, PF₆⁻, CIO₄⁻, CHO₂⁻, CF₃CO₂⁻, CF₃SO₃⁻, CH₃CO₂⁻, ArCO₂, CH₃SO₃⁻, p-tolylSO₃, HSO₄⁻ and H₂PO₄⁻ and B(Ar)₄⁻.

12. The method of claim 10, wherein R₂ is H and each X is halogen.

13. The method of claim 10, wherein R₂ is H; and each X is H.

14. The method of claim 10, wherein the catalyst has the formula Ru(R₃R₄SO)₄Xₘ.

15. The method of claim 10, wherein y has a value of 1, 2 or 3, and further wherein each L present is independently chosen from the general formula (PR₅R₆R₇), each of R₅, R₆, and R₇ being independently chosen from alkyl, aryl, alkoxy, and aryloxy.

16. The method of claim 10, wherein y has a value of 1, 2 or 3, and further wherein at least one L is (PPh₃) or CO.

17. The method of claim 10, wherein R₃ and R₄ are independently chosen from C₁ to C₆ alkyl, aryl, alkoxy, and aryloxy.

18. The method of claim 10, wherein m is 2 and X₂ is H₂.

19. The method of claim 10, wherein each X is a halogen.

20. The method of claim 10, wherein the catalyst is chosen from Ru(DMSO)₄Cl₂; Ru(DMSO)₄H₂; Ru(DMSO)₄HCl; Ru(PPh₃) (DMSO)₃O₂; Ru(PPh₃) (DMSO)₃H₂; and Ru(PPh₃) (DMSO)₃HCl.
